(19) United States
(12) Patent Application Publication
(10) Pub. No.: US 2002/0137085 A1
(43) Pub. Date: Sep. 26, 2002

(54) Multiplexed generation of chemical or physical events

(76) Inventor: Steven S. Herrick, Los Altos, CA (US)

Correspondence Address: Charles D. Holland, Morrison & Foerster LLP, 755 Page Mill Road, Palo Alto, CA, 94304-1018, US

(21) Appl. No.: 10/084,410

(22) Filed: Feb. 25, 2002

Related U.S. Application Data
(63) Continuation of application No. PCT/US00/23289, filed on Aug. 25, 2000.
(60) Provisional application No. 60/151,158, filed on Aug. 27, 1999.
(60) Provisional application No. 60/174,969, filed on Jan. 6, 2000.

(51) Int. Cl. C12Q 1/68; C07H 21/04

(52) U.S. Cl. 435/6; 536/25.3

(57) ABSTRACT

Methods and devices are provided for producing dense arrays of chemical entities. A substrate comprises a plurality of microlocations having microelectrodes connected to a network for connection to a computer to control the voltage and polarity at each of said microelectrodes. Means for producing electrically charged microparticles comprising at least one chemical moiety produce a mist of the particles which is directed to the surface of said substrate, where the microparticles are captured by microlocations of lower potential. By providing chemical moieties concurrently or sequentially, oligomers may be formed or small organic compounds synthesized. The resulting arrays may be used for screening samples for specific binding entities.

### RELATED APPLICATIONS

This application is a Continuation of PCT/US0023289, filed Aug. 25, 2000, which claims the benefit of priority to U.S. Provisional Application Serial No. 60/151,158 filed Aug. 27, 1999, and to U.S. Provisional Application Serial No. 60/174,969 filed Jan. 6, 2000, each of which are incorporated herein by reference

### INTRODUCTION

1. Field of the Invention

The field of this invention is manufacturing of a multiplexed device for performance of a multiplicity of chemical or physical operations at microlocations to synthesize, assay or perform other operations at different sets of microlocations by means of charged aerosolized particles and charged microlocation sites.

2. Background

Biotechnology has created the need to perform myriad operations in the unraveling of the genome, identifying polymorphisms, sequencing DNA, identifying alleles, comparing sequences between species and among individuals of the same species, relating particular sequences, particularly sequences involving single nucleotide polymorphisms, to traits, identifying pathogens and pathogen strains, and the like. Concurrently, combinatorial chemistry has greatly expanded the ability to synthesize large numbers of diverse compounds, but in relatively small amounts. These interests have created a need for devices and methods, which allow for screening large and frequently diverse mixtures, as well as performing numerous operations and obtaining information about the mixture.

One area of particular interest is the sequencing and screening of DNA mixtures. Toward this end, substantial effort has been made to build microarrays of oligonucleotides, where the sequence at each site or microlocation of the oligonucleotide is known. These microarrays are designed to have a high density of oligonucleotides, allowing for a compact surface, which is contacted with the nucleic acid sample. In order to provide as much diversity as possible, where the amount of target DNA may be low, one wishes to have as small a volume as possible for contacting the microarray. Different lithographic techniques borrowed from integrated circuit manufacturing have been developed to prepare the oligonucleotides in situ. These processes are difficult for this purpose, since at each stage for each nucleotide, optical masking is required to prevent reaction at some microlocations, while allowing for reaction at other microlocations.

Also, there is interest in being able to synthesize a large population of compounds based on a predetermined chemical motif. By using a few initial reactants and modifying them by using different reagents, within a synthesis of a few stages, one can realize a large diversity of compounds. The field of combinatorial chemistry has greatly expanded the ability to prepare large numbers of compounds. Various techniques have been developed to be able to differentiate the different compounds.

In addition, there is interest in the ability to screen numerous events, such as the binding between proteins, affect of candidate compounds on binding events or enzyme activity or the like, interactions between different compositions, such as catalytic activity, etc. For these purposes, where one is interested in screening a large population for one or more activities or properties, it is desirable that only a small amount of the various members of the population be required and that the events be performed in small volumes. By having small volumes, there is a low expenditure of reagents, concentrations may be relatively high and the reactions are fast, due to the short distances traversed for collisions between molecules.

For all of these purposes there are certain universal needs: the ability to differentiate between individual members of the group; the ability to direct particular moieties to a particular site; and the reliability of the process. There remains substantial interest in providing methodologies and devices which allow for the synthesis of diverse compounds, whether oligomers or non-oligomeric compounds and for assaying complex mixtures or large numbers of diverse molecules.

### RELEVANT LITERATURE

U.S. Pat. No. 5,605,662 describes preparing arrays using electrophoresis. Oligonucleotide array preparation is described in U.S. Pat. Nos. 5,744,305 and 5,831,070. Combinatorial synthesis of small organic molecules is described in U.S. Pat. No. 5,789,172. The use of aerosols is described in U.S. Pat. Nos. 5,066,512 and 5,103,763, as well as Pennebaker, Proceeding of the S.I.D. (1976) 17/4:160-168; Goldowsky, SID 90 Digest, 80-82; and Chen et al., J. Aerosol. Sci. 1995, 26:963-977

### SUMMARY OF THE INVENTION

Methods and devices are provided for performing multiplex and multi-step reactions at addressable sites. Devices comprise a plurality of microlocations on a substrate, which microlocations can be independently addressed to provide an electrical potential pattern. Reactants and/or reagents are directed to individual sites by using uniphase charged microparticles in an aerosol and providing polarized microlocations oppositely charged to the microparticles to attract the microparticles to the lower potential locations. Also, microlocations in close proximity to the attracting microlocation may be polarized with the same charge as the microparticles to enhance the accuracy with which the microparticles are directed. The charged microparticles are captured by the field created by the microlocation of lower potential and directed to the microlocation of lower potential. One or more events can be performed consecutively or concurrently at each microlocation, where one or more components of the reaction may be provided at each microlocation, followed by delivering additional reactants and/or reagents to individual microlocations. Oligomeric arrays may be synthesized and used for interrogating complex samples or individual assays performed, where different agents are present at different microlocations.

A particularly useful device having a plurality of microlocations is a memory chip as found in computers or as otherwise utilized by the electronics industry. The memory chip has a number of cells that are the microlocations discussed herein, and the chip is modified so that it has a hole partially or completely through the passivation layers covering each cell at which an oligomer is to be grown. Each cell of the chip is addressed by activating a column and row and placing the desired potential on the metal portion of the cell. A memory chip so configured can have a very high density of oligomers on the chip (over 500,000 oligomers on the memory chip) while requiring many fewer connections to the memory chip (only thirty-two connections for a memory chip having about 256,000 cells), and moreover the oligomer at each of the 500,000 or more cell locations can be a different sequence if desired.

A particularly useful system for reactant deposition and oligomer growth comprises a charged microparticle generator, a modified memory chip, and an electronic system that places a desired potential on cells by activating rows and columns in the modified memory chip and placing a desired potential at selected memory cells. The charged microparticle generator produces an aerosol of microparticles of reactant, each of which microparticles carries an electrostatic charge on its surface. The aerosol from the charged microparticle generator is directed at the surface of the memory chip, and the potential difference between the microparticles and selected cells on the memory chip causes microparticles from the aerosol to deposit at the selected cells. In the absence of a sufficient potential difference between the microparticles and memory cells, the aerosol flows around the memory chip, and microparticles entrained in the gaseous stream do not deposit onto the memory chip.

Among other factors, the invention is based on the technical finding that an array of different oligomeric nucleotide sequences or other chemical sequences can be formed by directing an aerosol of electrostatically charged microparticles having a first potential at the surface of a modified memory chip which has selected microlocations on the memory chip at a second potential that provides a sufficient attraction between the electrostatically charged microparticles and the selected microlocations that the microparticles depart from the gaseous stream of the aerosol and deposit at the selected microlocations. The use of a memory chip in the deposition system of this invention provides an economical and reliable method of making customized and/or individualized arrays of genetic or other sequences on a substrate at very high density. An array of this invention can have much higher oligomer density than can an array that requires space-consuming and unreliable individual electrical lead paths to each of the microlocations (500,000 lead paths and connection sites if 500,000 microlocations are to be addressed). Further, the chip can be an available off-the-shelf memory chip that is modified slightly, and the chip therefore does not have to be a custom-designed chip as is required in other systems. These an other technical findings and advantages are apparent from the discussion herein.

### BRIEF DESCRIPTION OF THE DRAWINGS

FIG. 1 is a diagrammatic view of a substrate with microlocations, according to this invention;

FIG. 2 is a diagrammatic view of a charged microparticle generator using a corona discharge for producing charged gas particles to subsequently charge microparticles produced with a nebulizer; and

FIG. 3 is a diagrammatic view of a charged microparticle generator using electrohydrodynamics to produce charged microparticles.

FIG. 4 is a schematic figure of a memory cell found in a typical bipolar RAM memory chip as used in this invention.

FIG. 5 is a schematic figure of a memory cell found in a typical MOS RAM memory chip as used in this invention.

FIG. 6 is a schematic figure of a memory cell found in a typical DRAM memory chip as used in this invention.

### DESCRIPTION OF THE PREFERRED EMBODIMENTS

The subject methods and devices allow for the synthesis of diverse dense arrays of oligomers or small organic or inorganic molecules, the performance of a plurality of chemical and physical events at different sites on a substrate, and the ability to interrogate individual sites to determine the occurrence of events. A basic device is employed having a substrate with a plurality of microlocations in close proximity, which are individually addressable by means of computer controlled individual electrodes which can be given a predetermined potential. Adjacent microlocations may have a different potential to enhance the specificity of the direction of the microparticles . Reactants and/or reagents (hereinafter referred to as “chemical moieties”) may be placed at individual sites by any convenient means. Additional chemical moieties are then brought to individual microlocations as charged aerosol microparticles, which are captured by the field created by the electrical potential at the microlocations and deposited at the site of the lowest potential microlocation. Depending on the nature of the chemical moieties involved, chemical reactions may occur, with or without the intervention of changes in temperature. Where a common chemical moiety is used, the entire substrate may be treated with the chemical moiety. The steps may be repeated as often as necessary, with individual or sets of microlocations being treated differently at different stages to provide the desired diversity of products or events.

A system of the invention can therefore comprise: (1) a charged microparticle generator; (2) a memory chip having multiple microlocations or cells with microwells (each of the cells being configured so that it is accessed by row and column address lines that are shared individually with adjacent cells); and (3) an electronic system such as a computer that sets the potential of each cell by activating selected row and column addresses and provides a desired potential to some or all of the activated cells. The charged microparticle generator is configured to provide an aerosol of electrostatically charged microparticles (e.g. droplets) that have little total charge (which is distributed on the surface of the microparticles) but large potential. The charged microparticle generator directs the electrostatically charged microparticles to the memory chip (or other substrate) at a velocity sufficiently slow to prevent deposition of the electrostatically charged microparticles in the absence of a potential difference between the electrostatically charged microparticles and the cell and wherein the velocity is also sufficiently slow to allow the electrostatically charged particles to deposit within microwells at cells where there is a sufficient potential difference to attract the electrostatically charged particles of large potential from the aerosol. (The term “large potential” is used to indicate that the electrostatically charged droplets have a potential that differs sufficiently from the potential of a microwell or electrode where the droplets are to be deposited that the droplets in fact deposit within the microwell under appropriate conditions as described herein. The droplets usually have additional electrons distributed across the surface of the droplets, providing a large potential compared to the ground or positive potential that is found within the memory chip.)

In describing the subject device, the fabrication of the substrate will be considered first. Secondly, will be considered the formation of the aerosol. Third, will be the formation of products and/or the assaying of events at the microlocations.

Fabrication of the addressable microlocation device

The addressable microlocation device upon which sequences are formed may be any memory chip that is capable of providing a sufficient potential difference between selected cells and electrostatically charged microparticles that the microparticles deposit at the selected cells and do not deposit at the remaining cells. For example, commercially-available RAM or ROM chips may be modified to make them suitable to grow sequence arrays. Typical RAM or ROM chips have passivation layers over the cells whose thicknesses make the chips themselves unsuitable for use in forming sequence arrays. However, these memory chips can be modified to make them suitable to grow arrays. In one example, a commercially-available RAM chip, which has a silicon oxide passivation layer over the aluminum portion of the memory cell and a silicon nitride passivation layer over the silicon oxide passivation layer, can be patterned in a single exposure step and etched to form microwells above or below each of the cells. Only one patterning step is required, and therefore each of the microwells is accurately aligned to a corresponding memory cell.

A memory chip has memory cells organized in an array formed on a substrate. Cells are accessed by providing the appropriate voltages to the rows and columns to which transistors of the cells are connected. A typical memory chip has a capacity of about 64 kilobits (“64 k”), 128 kilobits (“128 k”), 256 kilobits (“256 k”), and about 512 kilobits (“512 k”) or more.

Because the cells are organized in an array that is addressed by activating rows and columns, a large number of reaction microlocations or cells can be accessed and their potential set using only, e.g. thirty-two connections as are present in typical 256 k memory cells. An arrangement of cells in an array and addressed via row and column address lines reduces the number of connections needed by a substantial extent. Otherwise, at least one conductive line per microlocation is required to address the individual microlocations, and a bond pad is also required for each conductive line to connect the array of microlocations to an electronic system that controls the electrostatic potential of each microlocation. The use of a memory chip using column and row address lines and controllers allows a much denser array to be formed on a chip, since only thirty two lines are needed to address e.g. 256,000 cells rather than 256,000 lines as needed for individual microlocation addressing.

Commonly-available memory chips may be modified to provide at least substantially all of the cells with microwells so that the desired sequences may be grown in each of the microwells. These chips include random access memory (RAM) such as: SRAM (static random access memory, which stores information depending on which of its transistors in its flip-flop circuit is activated); DRAM (dynamic random access memory, which stores information by placing an electrostatic charge on a capacitor plate); EDO RAM (extended data output RAM, which contains a latching circuit); and SDRAM (synchronous DRAM, which is synchronized to a computer's clock) such as: JEDEC SDRAM (designed to meet the guidelines set by the Joint Electron Device Engineering Council); CDRAM (cache DRAM); RDRAM (Rambus DRAM); ESDRAM (enhanced SDRAM); DDR-SDRAM (double data rate SDRAM); and SLDRAM (SyncLink DRAM). Each of these RAM memories has a structure that is maintained at an appropriate potential during programming and thus can attract electrostatically charged microparticles such as charged reactant droplets, as described later.

Other commonly-available memory chips include EEPROM (electrically erasable and programmable read-only memory) and EPROM (electrically programmable read-only memory) chips. These chips also have a structure such as a floating gate or line that maintains a potential during or after programming that can be used to attract electrostatically charged microparticles.

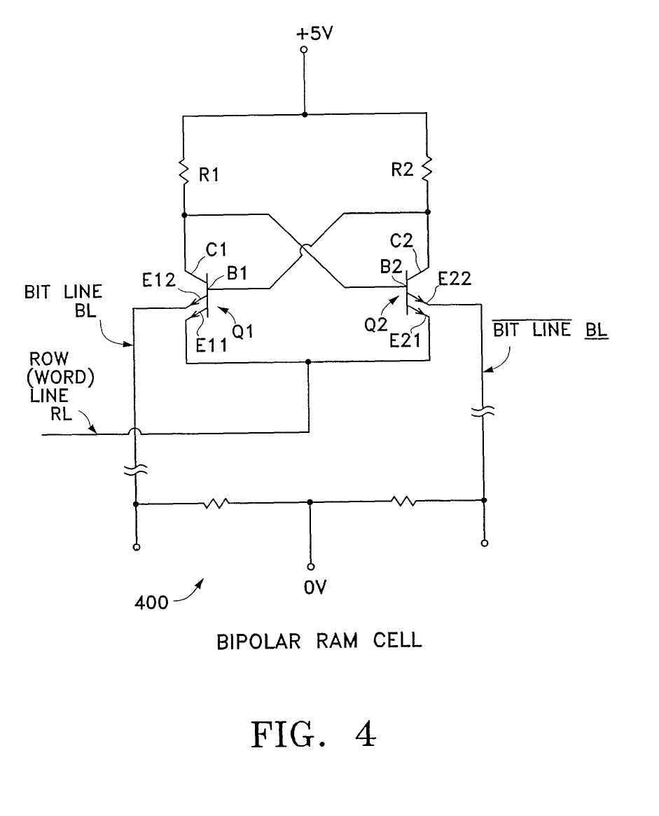

FIG. 4 illustrates a memory cell 400 of a typical bipolar RAM chip that can be used in one of the preferred embodiments of the invention. Two multi-emitter transistors Q1 and Q2 are connected to two load resistors R1 and R2 to form a flip-flop circuit. One emitter from each transistor (E11 and E21) connect together and form the row line input RL. Emitter E12 on Q1 is the bit line output BL, and the other emitter on Q2 is its complement BL.

Assuming this cell is in logic state 1, Q1 is latched on and Q2 is latched off. Collector C1 of Q1 is therefore at 0V, while base B1 of Q1 and line 401 are at +5V. Base B1 or a convenient place along metal line 401 is therefore an appropriate location to position a microwell in which droplets are to deposit. There is a large potential difference between the negative potential on the droplet created by the electrons distributed across the surface of the droplet and the positive potential at the base B1 of Q1 when data having a logic value=1 is written to this cell.

When data having a logic value=0 has been written to this cell, base B1 is maintained at 0V because transistor Q2 is latched on. There would not be a sufficient potential at base B1 to attract droplets from the aerosol to a microwell positioned above base B1. (While a voltage of +5V has been used for illustrative purposes, the voltage should be selected to provide a sufficient potential difference between the droplets and the base to attract the droplets from the aerosol.) The use of such a self-latching circuit is preferred, since it is not necessary to constantly restore the potential at the microlocation on the memory chip by refreshing the data (as explained later).

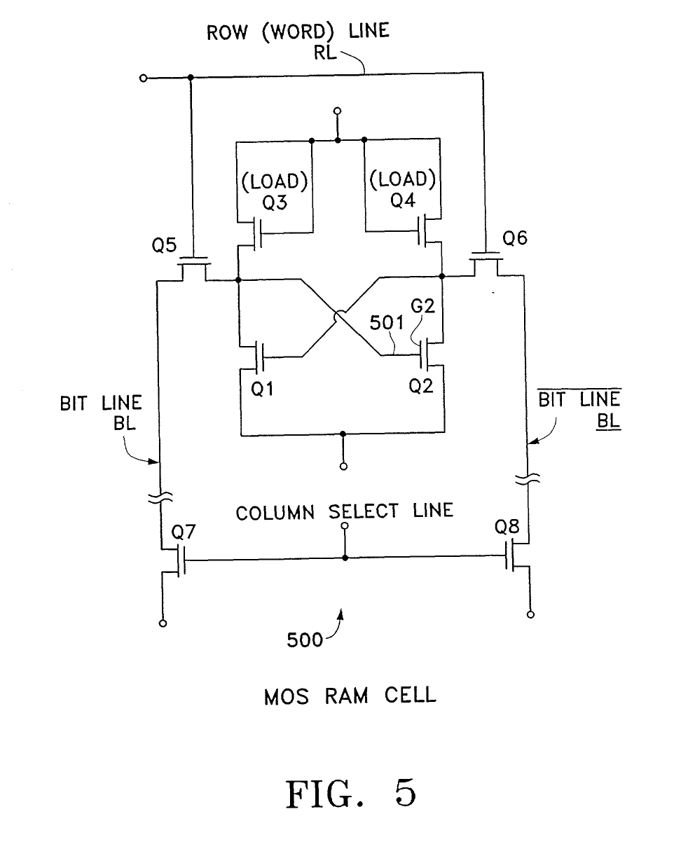

FIG. 5 illustrates a MOS RAM cell 500 from a typical MOS RAM chip used in another preferred embodiment of the invention. In this circuit, MOSFETs Q1, Q2, Q3, and Q4 form the flip-flop. Q1 and Q2 are the switching elements, and Q3 and Q4 work similarly to load resistors R1 and R2 illustrated in FIG. 4. MOSFETs Q5 and Q6 act as transmission gates that isolate or connect the outputs of the cell with the bit lines BL and BL. Q5 and Q6 are activated by the row line RL. Q7 and Q8 also act as transmission gates that isolate the bit lines from or connect the bit lines to the sense amplifier inputs of the memory chip.

When data is written to the cell, gate leads of Q5, Q6, Q7, and Q8 are held positive by selecting the cells using its row and column lines. Q2 is turned on and Q1 off by placing BL at logic value=1 and BL to logic value=0. The flip-flop latches in this state when Q5, Q6, Q7, and Q8 are turned off. Gate G2 on Q2 and metal line 501 are held above the threshold value of voltage by load transistor Q3. There is a large potential difference between the electrostatically charged droplets and the positive potential at gate G2, and thus the microwell can be positioned above the electrode for gate G2 or at a convenient spot along metal line 501.

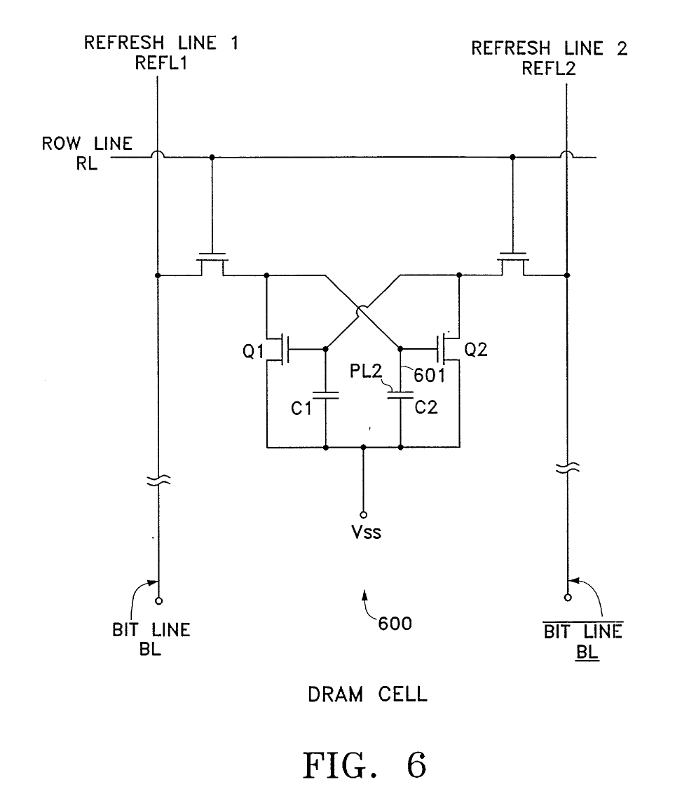

FIG. 6 illustrates a memory cell 600 as found in a typical DRAM memory chip used in another preferred embodiment of the invention. In this cell, logical data is stored in capacitors C1 and C2. When data having a logic value=1 is written by selecting the row line RL and setting bit line BL to the voltage for logic value=1 (e.g. +6 volt) and BL to logic value=0 (e.g. +1 volt), capacitor C1 charges to a capacitance equal to logic value=0 (VSS=0V), and keeps transistor Q1 turned off. Capacitor C2 charges to a capacitance equal to logic value=1, and transistor Q2 is turned on. After data has been written, row line is shut off, and capacitor C2 retains a potential of logic value=1.

A microwell is therefore positioned above plate PL2 of capacitor C2 or a convenient spot along line 601, since the potential difference between the positive potential of the plate and the negative potential of the electrostatically charged particles is high. Capacitance leaks off of the capacitors, and the data is refreshed periodically by turning on row line RL and supplying refresh voltage to refresh lines REFL1 and REFL2 to recharge the capacitors. Once the cell has been charged with data, it may be necessary to rapidly or continuously refresh the data stored in capacitors C1 and C2 while depositing droplets, since the charge transferred from the droplets to plate PL2 quickly neutralizes the positive potential of the plate where the liquid from the droplets contacts the plate.

Generally, droplets are deposited after the memory chip is programmed, although droplets may be deposited while the memory chip is being programmed or when data is read by selecting a suitable location in the circuitry of the memory chip as is apparent to a person of ordinary skill in this art based on the disclosure herein.

In each instance, the position selected for droplets to deposit in the memory cell is a position for which there is a unique potential once data is written and for which there is a sufficient potential difference between the electrostatically charged microparticles and the position that the microparticles are attracted from the aerosol and to the position.

Instead of attracting microparticles to a microlocation, a microlocation may be maintained at the same potential as the electrostatically charged microparticles to repel them from that microlocation. This helps to assure that unwanted microparticles do not deposit at that microlocation.

Other memory chips that may be used in the practice of the invention include: CCD memory (charge-coupled device memory) and content addressable memory, each of which stores information by placing an electrostatic charge on a capacitor plate; PROM (programmable read-only memory) chips, ROM (read-only memory) chips and PLA (programmable logic arrays), each of which stores information by connecting a cathode to the bit (column) line of the array or leaving it open. Although these devices generally have a set program that does not change during use, these memory chips are useful to fabricate arrays when it is desirable to form identical compounds in specific locations on a chip.

As discussed previously, microwells are formed through the passivation layer or layers over top of the metal or doped silicon or polysilicon whose potential is used to attract the electrostatically charged droplets from the gaseous stream. All microwells can be formed as the result of a single lithographic step and subsequent etch to remove some or all of the silicon nitride and/or silicon oxide of the passivation layers. In some applications, it is desirable to leave some of the passivation layer to protect the underlying structure that carries the potential. Enough of the passivation layer is removed so that the electric field extending through any remaining passivation layer has sufficient strength to attract electrostatically charged microparticles from the gaseous stream carrying the microparticles.

Other addressable microlocation devices besides memory chips may be formed on a substrate. The substrate may be of any convenient size, where the array of microlocations is typically 95% or less of the surface space of the substrate, usually at least about 50%, more usually at least about 75%, of the substrate surface. Generally, the substrate will be at least about 1 mm², more usually at least about 2 mm² and generally not more than about 5 cm², more usually not more than about 2.5 cm². Of course, larger substrates could be used and one could have a plurality of separate devices made on the same substrate so as to simultaneously perform a plurality of operations. In most cases, however, it will be desirable to miniaturize the device, which will result in a smaller instrument, the use of less of the chemical moieties, and smaller areas of detection. On the other hand, macroarrays may find use, where one wishes to produce isolatable amounts of material, have a strong signal, or the like, which may require larger microlocations and larger substrates.

In designing the device, there is a plurality of microlocations, each one having a functioning microelectrode. The microlocation will be formed to have an electrically conducting base and sufficient depth to accommodate the volume of liquid to be placed at the microlocation. The volume of the well will usually be at least about 10 nl, more usually at least about 20 nl, and not more than about 5 ml, usually not more than about 2 ml, depending upon the nature of the operations, the number of different reagents which must be delivered to the well at each stage, the nature of the reagents, and the like. Desirably, the volume will be in the range of about 20 nl to 500 μl. The cross-sectional area will usually be in the range of about 20 to 10⁴ μm². While larger microlocation volumes may find use for particular applications, e.g. synthesis, these volumes will usually not exceed about 5 ml, usually not exceeding about 1 ml.

Generally, the microlocations will be placed as close together as possible without shorting between two adjacent microlocations. The number of microlocations per cm² will generally be in the range of about 100 to 10⁶, more usually about 100 to 10⁵.

The microlocations may be formed by microlithographic and/or micromachining techniques. A semiconductor substrate, e.g. silicon, is conveniently employed to provide for the electrical connections to the microlocations. The semiconductor substrate may be coated by an insulating layer, such as glass, ceramic, plastic, silicon dioxide or the like, and the individual metal bases separated by insulation barriers to prevent electrical conductivity between the metal bases of the microlocations.

In fabricating the device, mask design and standard microlithographic techniques may be used. The base substrate is conveniently a 1 to 2 cm² silicon wafer or a chip approximately 0.5 mm in thickness. The silicon is first overcoated with a 1 to 2 μm thick silicon dioxide insulation coat, which may be applied by chemical vapor deposition. A metal layer is then deposited by vacuum evaporation. The choice of metal will depend on the use of the device and the compatibility of the metal with the chemical moieties with which it will be in contact.

While aluminum is very convenient and has found extensive use in other situations, for many of the desired applications, it will not be acceptable and a more inert metal will be required or a protective coating is required. Coatings which may find application are organic electrically conducting coatings, such as metal or carbon containing coatings, polycyanoethylenes, polyacetylenes, etc. or, as appropriate, insulating layers, such as silicon dioxide. The insulating layer will be thick enough to protect the metal electrode from the chemical moieties used in the operations. In selecting a metal electrode, besides chemical inertness, other considerations for the choice will be ease of deposition, uniformity of the layer, ease of processing, cost and interaction with the selected insulating layer, ease of replacement, ease of formation, and the like. Metals of interest include copper, nickel, tungsten, lead, mercury, iron, cobalt, bismuth, vanadium, tungsten, silver, tin, platinum, palladium, zirconium, iridium, etc. The metal layer may be as a result of vacuum deposition, electroplating, chemical or electrical reduction, thermal decomposition, etc.

Instead of leaving a portion of the passivation layer to protect the potential-carrying structure, all of the passivation layer above or below a cell may be removed, and a thin protective layer may be coated onto the potential-carrying structure. One may provide for a reactive metal coating, e.g. aluminum or magnesium, which is then reacted with a metal salt, to reduce the metal and leave a layer of the metal from the salt in place of the reactive metal coating. One may provide for electrochemical reduction of a metal salt at the surface of the well. Alternatively, one may provide for thermal or photolytic decomposition of metalloorganic molecules, where the metal is plated onto the surface. Metalloorganic molecules include metal carbonyls, metal aromatics, metallocenes, and the like. Special techniques may be employed to ensure adhesion to the insulating sublayer, depending on the metal and the mode of application. Where a metal electrode has been formed at the pel sites, the other metal source may be delivered by any convenient means, such as pins, ink jets, aerosols, etc., once the electrode pattern has been established.

The thickness of the passivation layer or protective layer may be selected to provide a self-regulating deposition mechanism that limits the number of microparticles that deposit into a microwell. The thickness of the passivation or protective layer is selected to provide a desired field strength within the microwell created by the cell that attracts only the desired volume of electrostatically-charged droplets before the microwell loses field strength to attract more droplets. For example, the potential-carrying structure (e.g. electrode) of the cell is charged to provide the desired potential within the microwell positioned above or below the cell. One electrostatically charged droplet is attracted from the gaseous stream, thus reducing the potential difference between the charged droplets and the well by the amount supplied by the electrostatically-charged droplet. One electrostatically-charged droplet carries little charge (despite having a large difference in potential from the potential-carrying structure of the memory cell), and thus a sufficient difference in potential between the droplets and cell remains to attract other droplets. This is especially true where the droplet can contact the electrode (either directly or through a layer over the electrode) and dissipate its charge. A second electrostatically charged droplet is attracted from the gaseous stream, further increasing the potential within the microwell if the droplet cannot dissipate its charge but also further increasing the volume within the microwell, further reducing the potential difference between the droplets and the microwell. As the number of droplets within the well increases and as the depth as well as the full breadth of the well is wetted by the droplets, the potential difference between the droplets and the microwell decreases to a level that the well no longer attracts droplets from the gaseous stream. Thus, the thickness of the liquid layer formed by the droplets acts to reduce the potential difference between the surface of the liquid layer and the droplets. Further, any electrostatic charge remaining in the liquid layer further acts to prevent additional droplets from depositing, since the liquid layer has a potential that is much closer to or identical to the potential of the droplets. The thickness of the passivation or protective layer is therefore selected to provide the desired attenuation of electric field so that only a desired amount of the electrostatically charged microparticles deposit within a microwell, thus providing a self-regulating mechanism to prevent overfilling the microwells.

Thus, the use of an insulating layer may serve an additional purpose in limiting the number of microparticles deposited at a single microlocation. To the extent that the microparticles cannot dissipate the static charge, the static charge of the microparticles can build up to offset the field created by the electrode, so as to no longer attract additional microparticles. In this way, the amount of liquid delivered to each location will be self-regulated, while the microlocations of the same polarity as the microparticles will inhibit microparticles associating with such microlocations. Also, by limiting the total number of particles which are directed to the microlocation of opposite polarity, the likelihood of spillover from one microlocation to another is minimized,

Desirably the conducting metallic layer will be relatively thin, generally less than about 1 mm, more usually less than about 0.1 mm, although the thickness of the conducting metallic layer is not critical to this invention. Where some erosion of the conducting metallic layer occurs, a thicker layer will be desirable.

The chip may now be overcoated with positive photoresist, masked (light field) with the circuitry pattern, exposed and developed. The photosolubilized resist is removed and the exposed metal is etched away, as appropriate. After removing the resist island, the metal circuitry is left on the chip. This will include the array of microelectrodes, which serve as the underlying base for the addressable microlocations and the connective circuitry, and may also include an outside perimeter of metal contact pads.

Using CVD (“chemical vapor deposition”), the chip is overcoated with a 0.2-0.4 μ layer of SiO₂ and then with a 0.1-0.2 μ layer of silicon nitride. The chip is then covered with photoresist, masked for the contact pads and microelectrode locations, exposed and developed. Resist is removed and the SiO₂ and SiN₄ layers are etched away to expose the metallic electrode layers and the contact pads. The surrounding island resist is then removed, with the connective wiring between the contact pads and the microelectrodes remaining insulated by the insulative layers. Instead or in addition to the metal wires, silverized epoxy may be used for contacting microelectrodes, pads or for providing other electrical connections.

The subject device may be processed further, depending upon the nature of the metal and the operations to be performed. In some situations, it may be desirable to add a functionalized polymer to coat the metal, where the polymer is electrically conducting under the conditions of the operations. Various polymers may find use, particularly polymers such as conjugated olefins, polymers comprising electron accepting and electron donating functionalities, or other organic polymers, which allow for electron transport from charged aerosols to the microelectrodes. The polymers may serve other functions, such as being functionalized with reactive groups, e.g. amino, thiol, hydroxyl, carboxyl, phospho, etc., where linking groups may be attached, which act to retain a compound, particularly a multistage product, at the microlocation. Linking groups will generally be from about 1 to 60, usually 2 to 30 atoms in the chain, where the chain may be comprised of carbon, oxygen, nitrogen, sulfur, phosphorous, etc., including ethers, amino, thio, phospho, etc. groups in the chain.

An electrically-conductive filler may be used to coat the floor or walls of the microwells formed in a memory chip. This filler is capable of bearing a potential that is very different from the potential of the electrostatically charged microparticles and can therefore provide a sufficient difference in potential between the electrostatically-charged microparticles and the microwells that the microparticles are attracted from the gaseous stream flowing around the memory chip. One such filler is disclosed in U.S. Pat. No. 5,700,398, while another is disclosed in U.S. Pat. No. 5,876,586.

The wells which are formed by the operation will generally be at least about 1μ, more usually at least about 2μ, in depth and usually not more than about 10μ, more usually not more than about 5μ, in depth. The cross-sectional area will usually be in the range of about 10 to 2500μ², more usually in the range of about 25 to 500μ². Conveniently, the volume of the well will be in the range of about 100 nl to 500μl. The microelectrodes will generally be separated by at least about 2μ, more usually at least about 5μ, preferably not more than about 100μ, more preferably not more than about 50μ. While much larger spacings are possible, it is desired to have as high a density of microlocations as is compatible with maintaining electrical integrity (no arcing between microelectrodes), delivery of chemical moieties and identification of individual microlocations, where such detection is required. One can provide for addressable groups to be directed to individual locations, so that one can detect the individual location by detectable labels, such as fluorescers, chemiluminescers, or other label with the appropriate sensitivity, which can be isolated at a particular site.

Formation of the Aerosol

At least one chemical moiety during a process, synthetic diagnostic, etc., will be delivered as charged aerosol particles, usually at least one chemical moiety at each stage of the operation, particularly where the process is a multistep synthetic process. Various devices are available for producing aerosols, including ultrasonic nebulizers, electrohydrodynamics, piezoelectric transducers, electrospray sources, nozzles, gas jets, condensation, etc. The liquid is introduced into the aerosolizer and the aerosol formed. Aerosol particles will generally be in the range of about 10 nm to 5μ, more usually in the range of about 20 nm to 15μ diameter. The density of the microparticles will generally be in the range of about 10³ to 10⁷, more usually from about 10⁴ to 10⁶ particles per ml. Desirably, the particles are substantially monodisperse, that is, at least about 80 weight % are within about 20%, preferably within about 10%, of the average particle size. Monodispersity may be achieved using impact plates, electrohydrodynamics, precipitation, etc. Due to evaporation, the initial size of the particles may be substantially reduced, usually less than about 50 vol %, preferably less than about 25 vol %. The flow rates of the carrier gas will generally be in the range of about 0.1 to about 50 lpm, more usually about 0.2 to 20 lpm. The velocity will generally be in the range of about 0.1 to 70 cm/sec. In some instances, the carrier gas will be stopped, allowing the microparticles to approach the microlocations by diffusion and electrostatic attraction

The ultrasonic, piezoelectric or sonic aerosolizers for producing the particles may produce some large particles, which may be removed by using hydrodynamic impingement on a flat plate at the exit of the aerosolizer or electrostatic precipitation. The microparticles in the mist may then be passed through a microparticle charging zone which is connected to a charged air supply. Charging may be as a result of corona discharge, high voltage with oppositely charged electrodes and alternating current, or ionizing radiation. The resulting charged particles are then passed through a nozzle. A second stage fine filter, using direct current electrostatic precipitation plates may be used in the area of the nozzle to produce a monodisperse microparticle size. The stream of particles may be directed at an angle including normal to or parallel to the microlocation substrate. Depending on the impingement angle employed, different mist velocities may be involved.

For electrohydrodynamic charge generation (U.S. Pat. No. 5,247, 842), the fluid is passed through an electrically charged nozzle, where sheath flow of a neutral inert gas may surround the fluid and a source of compressed air is provided around the sheath to further direct the particles exiting the electrically charged nozzle. Liquid feed rates may be in the range of about 0.05 to 5μl per min. The flow of microparticles is directed toward an oppositely charged plate having a large orifice directly opposite the nozzle, so that the microparticles flow past the plate and may then be directed to the microlocations.

The conditions under which the charged particles may be formed are conventional and will be governed by a number of considerations, which include the electrical conductivity of the liquid, viscosity, surface tension, the desired size of the particles, the density of the particles, the solvent, the temperature at which the mist is formed and charged, the manner in which the charged microparticle mist is formed, and the like. The temperature of the mist will generally be in the range of about 4 to 60° C., more usually in the range of about 4 to 40° C. Conditions for forming monodisperse charged particles are described by Chen, et al., J. Aerosol. Sci. 1995, 26:963-977.

Various gases may be used as the mist carrier, particularly inert gases, such as air, carbon dioxide, nitrogen, helium, argon, or other convenient gas. Depending on the solvent for the microparticles, the gas may to varying degrees include the solvent as a vapor, where the solvent vapor pressure may initially be at least about 10% of the vapor pressure of the gas and up to saturation of the gas at the temperature of the mist. To avoid condensation on the microlocations, the substrate or memory chip may be maintained at a temperature higher than the mist temperature during deposition, generally at least about 2° C., more usually at least about 5° C. and generally not more than about 10° C. higher. By keeping the temperature differential small and the gas having a high vapor pressure of the solvent, not only will the microparticles retain at least a substantial portion of their liquid phase, but the microlocations will also be less subject to evaporation. Generally, elevated pressures will not be employed, although less than 1 atm increase in pressure may find application during the operation.

The mist is then directed over the microlocations. Depending upon the microparticle average size, a perpendicular mist will be at a velocity of less than about 70 cm/sec. to avoid non-specific adherence to the microlocation plate. The flow of the mist may be controlled by airflow, mild vacuum, the impinging of the mist stream onto the microlocation plate, or using a very slow gas stream or very low vacuum, or terminating any driving force directing the particles in any particular direction, where the mist may be substantially stationary. A description of a forced air supply may be found in U.S. Pat. No. 5,103,763. The smaller the target microlocation, the slower the flow of the mist will generally be. When having mist flow perpendicular to the microlocation plate, the mist will spread from the site to which it is directed across the surface of the microlocation plate. Microlocation plates of about 1 cm² can be covered with a single perpendicular mist flow toward the center of the microlocation plate. The mist may be as slow as about 0.1 cm/sec, or slower, usually not exceeding about 70 cm/sec. By providing for a cover plate over the substrate leaving a small gap between the cover plate and the substrate, the mist may be maintained in proximity to the microlocations and be subject to the electrical field produced by the microelectrodes. Generally, the spacing between a cover plate or other ceiling and the substrate surface will be at least about 100μ, and may be 500μ or more.

The nozzle through which the mist passes may have a length smaller than the dimension of the microlocation area of the substrate, usually not less than about 20% of the area, more usually not less than about 50% of the dimension, and usually not greater than about 200%, more usually not greater than about 150%. Where less than the entire location receives and captures the mist, the nozzle may be moved from site to site, until the entire microlocation area has been subjected to the mist. A precipitator plate may be used as the nozzle outlet to enhance the monodisperse character of the mist.

The liquid used to form the charged particles may be any liquid which is capable of carrying a charge and dissipating the charge to an electrode, which for the most part will be water, polar organic solvents, e.g. dimethyl formamide, nitrobenzene, trifluoroacetic, acid, hexamethylphosphoramide, acetonitrile, formamide, ethylene glycol, trimethylamine, etc. and mixtures thereof. The solvent will be selected in accordance with the operation being performed, so as to support the operation.

In operation, adjacent microelectrodes will preferably have varying potentials relative to an appropriate reference, so as to create a field about the microlocation plate. One may provide for alternating rows of microelectrodes, where the microelectrode beginning at each row starts with the opposite potential of the preceding row and the potential alternates along the row. In this way, each microelectrode will be surrounded by four microelectrodes of opposite potential.

The substrate may be provided with a heating and/or cooling source, such as a heat transfer plate, heat transfer coils, infra-red lamps, etc., where heat may be transmitted to specific microlocations to enhance reactions or heat dissipated from specific microlocations. The substrate may also be provided with a source of light, e.g. a laser, which may be directed to individual microlocations, portions of the microlocation area or the entire microlocation area, to permit photoreactions at the microlocations. The optical system will vary with the needs of the operations at the microlocations.

Formation of Products and Assaying Events at the Microlocations

The subject system allows for great variety of operations and the use of diverse chemical moieties. The subject system may be used for the synthesis of oligomers, such as oligonucleotides and oligopeptides, in accordance with conventional ways. Simple organic molecules may be synthesized using techniques associated with combinatorial chemistry. Since each microlocation is individually addressable, one can record the individual steps using a computer, so that the synthetic record will be known for each microlocation. In addition, one can carry out assays, determining various events, evaluating the activity of candidate compounds having biological activity, detecting the presence of a ligand or receptor of interest, and the like. Samples, candidate compounds, and chemical moieties may be delivered as an aerosol or may be initially introduced into the microlocations and additional stages of the operations performed, or may be prepared in situ. Aggregations of molecules may be distributed as a mist, including nucleosomes, liposomes, cells, organelles, nuclei, chromosomes, plasmids, double minutes, etc. In this way one can check to see the effect of candidate compounds on physiological processes, for example, measure changes in pH, calcium transport, etc.

Various compositions may be introduced into the microlocations for a variety of purposes. Polymeric compositions may be present to which chemical moieties may be bound, covalently or non-covalently. Various beads or sols may be added to the microlocations to serve as the site for the reactions, where the particles are functionalized for receiving the next member in the synthesis. Beads or sols may include latex beads, glass beads, porous glass beads, carbon sols, colloidal metals, e.g. gold, and the like. As illustrative, for oligonucleotide synthesis, four different beads can be provided with the four different nucleotides bonded to the beads by linkers and be ready for further reaction with the next successive nucleotide. Similarly for amino acids, except that up to 20 different amino acids may be initially present. Beads are commercially available and will generally be of a size in the range of about 5 to 500μ diameter.

In preparing oligomers, normally a protected or blocked reactant is employed, e.g. a protected nucleotide or amino acid, where the site of protection is also the site for the addition of the next monomer. In carrying out the synthesis of the oligomers, one can direct individual monomers as microparticles to the appropriate microlocations for reaction with unblocked terminal monomers, where the unblocking is done by a bulk reagent or one may direct the unblocking reagent as microparticles to the appropriate locations and then add the appropriate monomer to the unblocked terminal groups as a bulk reagent.

For preparing oligonucleotides, various chemistries may be employed. See, for example, U.S. Pat. Nos. 5,436,327, 5,831,070 and 5,872,244. In carrying out the synthesis of oligonucleotides, protected phosphoramidites, phosphite esters or triphosphates may be employed. With the phosphoramidites and phosphite esters, no additional catalyst is required, while for the triphosphates, an enzyme is required, conveniently a terminal deoxytransferase. In the former two cases, the microlocations are functionalized, having a reactive amino group as a result of having beads in the microlocation wells or having an adhering functionalized polymer, which provides the amino groups. To initiate the synthesis of the oligonucleotide, a. mist is prepared of a phosphoramidite reagent in an appropriate organic solvent, conventionally acetonitrile, by itself or in combination with another organic solvent, particularly one having an elevated boiling point as compared to acetonitrile, such as methyl pyrrolidone, trimethyl phosphate, or N-methyl pyrroleacetonitrile. The composition may include a small amount of an electrically conductive substance, less than 1 vol %, usually less than 0.1 vol %, and depending on the nature of the additive, less than about 0.01 vol % or its weight equivalent, to change the characteristics of the microparticles. The additive may be water, glacial acetic acid, dimethylaniline, N-methyl pyridine chloride, or other compatible additive which will not adversely affect the chemistry and allow for dissipation of the charge of the microparticle. For detritylation, trichloro- or trifluoroacetic acid, zinc chloride, or other acidic agent may be used in an appropriate solvent, such as dichloromethane, dibromomethane, nitromethane, etc., combinations thereof, and the like, where a trace of moisture may be present to provide ionic species.

Instead of protective groups, which require chemical removal, one may employ protective groups which may be removed photolytically, thermally or electrolytically. Many of these groups are described in U.S. Pat. Nos. 5,744,305 and 5,889,165. The photolabile groups are benzyl ethers, carbonate mixed anhydrides or urethanes, where the phenyl group is substituted with 2-nitro and desirably, 4,5-dimethoxy, where the remaining sites of the benzene ring may be further functionalized. In preparing oligonucleotides, the ether or ester of the 3′-hydroxyl of the deoxyribophosphoramidite is employed. After adding the next nucleotide to the growing chain, the 3′-hydroxyl may be deprotected by irradiation with light in the range of about 350-375 nm at an intensity in the range of about 5 to 20 mW/cm². For electrolytic removal, see for example, WO 98/01221, inventor Donald Montgomery, assigned to Combimatrix Corp., which disclosure is incorporated herein by reference.

In place of chemically reactive groups, which do not require ancillary reagents for reaction, one may use reactants which can be linked enzymatically. See, for example, U.S. Pat. No. 5,872,244. Terminal deoxytransferases are used with an initiating substrate of a free 3′-hydroxyl and a nucleotide 5′-triphosphate with a removable 3′-protecting group. Protecting groups include carbonitriles, phosphates, carbonates, carbamates, esters, ethers, borates, nitrates, sugars, phosphoramidates, phenylsulfenates, sulfates and sulfones, where the preferred deblocking reagent is Co⁺² in a buffer, such as dimethyl arsinic acid, Tris, MOPS, etc.

Preparing oligopeptides generally follows the same pattern as the preparation of the oligonucleotides. A substrate having a reactive functionality is present in the microlocation for initiating the reaction. The functionality can be provided with a photolabile link or a chemically reactive link, which may be chemically cleaved. Amino acids may be protected with photolabile protecting groups, as described above, and the carboxyl activated for reaction with a free amino group. Activation may be achieved with the N-succinimidyl ester, mixed anhydrides, carbodiimides, etc. Usually an aqueous solvent will be employed. The reaction mixture is incubated and the protective group may then be removed chemically or photolytically.

It is frequently desirable when preparing oligomers to cap any unreacted free groups, such as the 3′-hydroxyl in oligonucleotide synthesis and the amino group in oligopeptide synthesis. The capping may be achieved using ester, amide or ether forming groups, such as organic acids, active halides, or the like, where the product of the capping is not cleaved during the steps of the oligomeric synthesis.

After each stage of the synthesis, the entire substrate may be washed to remove unreacted reagent and solvent from the previous step. Washing will usually be with an appropriate medium to remove organic solvents and/or unreacted reactants present at the microlocations. Depending on the efficiency of the washing, one or more washings may be performed with the same or different washing medium.

One may also use the subject invention for performing syntheses. Either the base, wall or beads introduced into the microlocation could act as a solid support for the synthesis. For a description of combinatorial chemistry, see, for example, U.S. Pat. No. 5,789,172. In this approach, labeled beads are used and the labels are removed at the end of the synthesis to determine the synthetic protocol used to produce the product on individual beads. At each stage, chemical moieties may be introduced as a mist or a bulk composition. Included among chemical moieties are synthons, which are small organic molecules, usually having a plurality of functionalities, so as add a unit for the synthesis, rather than a small reactive moiety, such as cyano, nitro or halo. Thus, small organic molecules (<1 kD) may be added to produce novel products and groups of novel products. With the subject invention, a record of the polarity of each site at the time of each addition of reagent would be kept, so that one would know the reagent, reactant and the conditions at each step for each microlocation. The only decoding would be reading the program for the particular microlocation. One could then screen the compounds in a variety of ways. If the free product is desired, the initiating moiety would be bonded to the solid substrate by a cleavable linkage. The linkage could be cleavable chemically or photolytically. The indicated patent provides for a number of linkages and reagents, which can be used, depending on the protocol and the product, for releasing the product from the solid substrate. Alternatively, the product could be left on the solid support.

Where the product is being prepared for a determination of biological activity, at the end of the synthesis, a biological reagent could be delivered to each site as a mist or bulk composition. For example, if one wished to determine the enzyme inhibitory effect of the product, the enzyme could be delivered to each of the microlocations and the enzyme and product incubated for sufficient time for the product to bind to the enzyme. A substrate, which provides for a detectable product in an appropriate medium for enzyme catalysis could then be introduced at each microlocation and the rate of reaction determined by monitoring the detectable product. As an illustration, if one was interested in a hydrolase, the hydrolytically cleavable bond could release a fluorescent product, which could be detected. Where NAD or NADP is involved in the enzymic reaction, the production of the reduced NADH or NADPH could be coupled with another reaction to produce a detectable dye. These techniques are well established in the literature and do not require elaboration here.

Alternatively, one may carry out assays, where the compound of interest may be initially introduced individually by any convenient means, pin transfer, ink jet , transfer from microtiter wells, mists and the like. As described above, again, the chemical moiety(ies) would be transferred to each of the microlocations, either the same chemical moiety(ies) or different chemical moiety(ies), as appropriate, as a mist or bulk composition. A capture compound could be employed, which would capture a member of the assay system, which provides, directly or indirectly, a detectable signal. One would then detect the detectable signal from each microlocation as an indication of the characteristic of interest for each compound of interest.

Characteristics of interest include enzyme activation and inhibition, binding to a receptor, particularly a surface membrane receptor, inhibition of complex formation, e.g. transcription factor complex formation, antioxidant activity, oxidative or reductive activity, activator or inhibition of a physiological process, e.g. angiogenesis and apoptosis, etc. For all of these purposes, reagents are available, where by using competitive assays, production of detectable compounds, destruction of detectable compounds, coupling with reactions, which provide a detectable compound, and the like, one may determine the effectiveness of a candidate compound for a characteristic of interest.

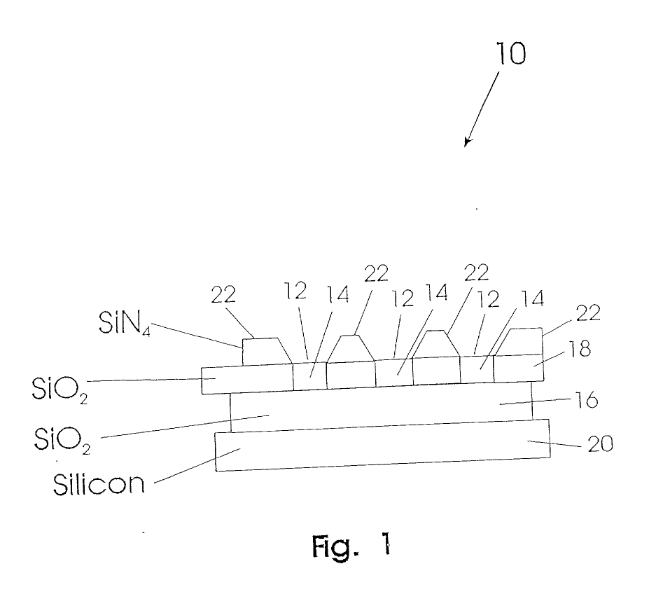

For further understanding of the invention, the figures will now be considered. FIG. 1 depicts a basic design of a device according to this invention, where each of the microlocations is self-addressable. The device 10 is microfabricated with three microlocations 12, which are equivalent for the purposes of this invention. While three microlocations are shown, it is understood, that the device would have a much greater number of microlocations, which could be organized in rows and columns as in a memory chip, about one or more axes of symmetry, on a disc, which would have a center feature for rotation, e.g. a registration cavity or other feature for affixing to a rotating device, etc.

An exemplary fabrication is as follows although a conventional memory chip is preferably used. The microlocations are formed in a base substrate material, e.g. 1-2 cm² silicon wafer about 0.5 mm thickness. The wafer is first overcoated with a 1-2 μm thick silicon dioxide insulation coat, which is applied by chemical vapor deposition (CVD). In the next step, 0.2-0.5 μm aluminum or other metal layer is deposited by vacuum evaporation. Depending on the metal, different techniques may be employed to ensure adhesion to the silicon. After overcoating with a positive photoresist, masked with the circuitry pattern, exposure and development, the photosolubilized resist is removed and the exposed metal etched away. The resist island is now removed, leaving the metal circuitry pattern on the wafer. This includes an outside perimeter of metal contact pads, the connective circuitry and the center array of microelectrodes, which serve as the underlying base for the addressable microlocations. Using CVD, the chip is overcoated with a 0.2-0.4 μm layer of silicon oxide and then with a 0.1-0.2 μm layer of silicon nitride. The chip is then covered with a positive photoresist, masked for the contact pads and microelectrode locations, exposed and the silicon dioxide and silicon nitride layers etched away to expose the aluminum contact pads and microelectrodes. The surrounding island resist is then removed, while the connective wiring between the contact pads and the microelectrodes remains insulated by the silicon dioxide and silicon nitride layers.

The contacts with the microelectrodes would be connected to a computer. The computer would control the potential at each microlocation, as well as the operation of the mist generator and the introduction of the chemical moieties and synthons into the mist generator or the bulk composition, recording each event as to each microlocation and each stage.

The microlocations 12 have metal bases 14, which may be any metal, depending on the chemistry to be performed at the microlocation. Conveniently it may be Al, an aluminum alloy, or other metal which may be vacuum deposited, reduced in situ, coated and then removed using photolithography, etc. The metal sites serve as the underlying microelectrode structures. In addition, various metals, by themselves or with conducting organic polymers, may be used as electroconductors, being formed in a variety of ways. See, for example, U.S. Pat. Nos. 5,700,398, 5,789,172, 5,804,563 and 5,876,586. An insulator 16 separates the metal microelectrodes 14 from each other. Insulators may include silicon dioxide, ceramics, glass, resist, rubber, plastic, etc. In each addition, each metal base 14, is further insulated from other metal bases 14, by an insulating wall 18, conveniently of silicon dioxide, but the other insulating materials indicated previously may also find use. The insulating layer 16 is supported by a silicon layer 20. Coated onto the silicon dioxide wall 18 is a silicon nitride layer 22, which is more chemically resistant than silicon oxide, so as to better withstand the conditions of the operations for which the device is used.

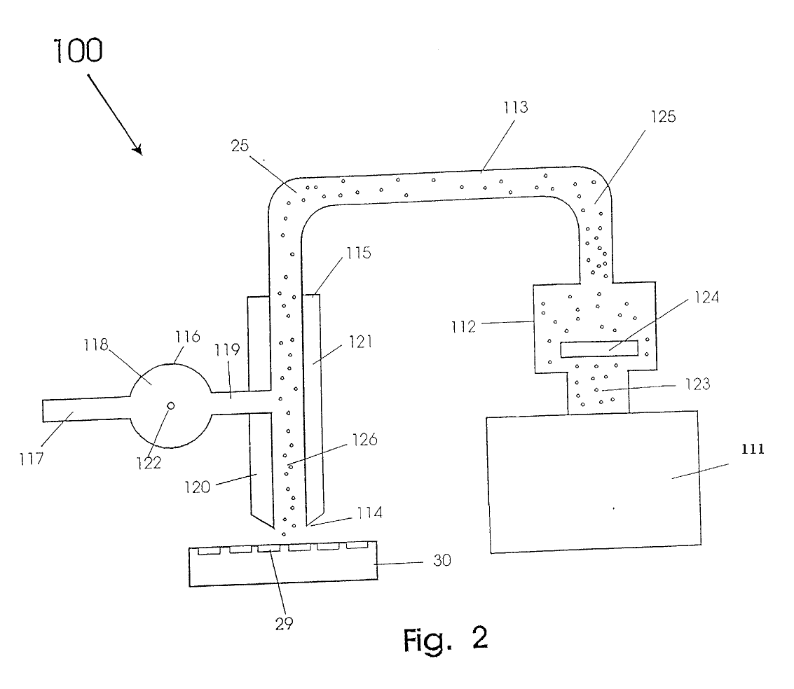

In FIG. 2 is depicted a device in which the mist is directed normally to the surface of the microlocation device. Referring to FIG. 2, the apparatus 100 which is illustrated is an A.C. field charging apparatus with which a charge can be placed upon fine droplets of a liquid. The apparatus 100 includes an ultrasonic mist generator 1 11, a mist conduit 113 terminating in a discharge nozzle 114 and charged air supply unit 116 which opens into said ion charging zone. The charged air supply unit 116 comprises an upstream forced air supply conduit section 119 opening into the droplet charging zone 115 of the mist conduit 113. The droplet charging zone 115 comprises oppositely-charged electrode plates 120 and 121, plate 120 being grounded and plate 121 being charged to about 1 kV AC, the alternating voltage frequency being about 5 kHz. The DC air ionizing zone 118 comprises a corona discharge element such as a corona wire 122, conveniently a 0.05 mm diameter tungsten wire, 5 cm long, having about 4,500 VDC applied to produce a corona current of 120 μa.

In operation, the ultrasonic mist generator 111 is supplied with an electrically conductive solution of a chemical moiety and operated at a frequency of about 1.7 MHz, where microparticles of about 3μ are desired. Greater or lesser frequencies may be used for smaller or larger microparticles.

The microparticles are forced through a jet nozzle 123 against a baffle plate 124 within the microparticle size separator 112 to cause larger microparticles to deposit by hydrodynamic impingement on the plate 124, while the desired smaller microparticles125 are carried around the plate 124 and enter the mist conduit 113. The uncharged microparticles are forced into the droplet-charging zone where they mix with and become charged by the ionized air introduced from the charged air supply unit 116.

The air supply unit 116 receives a supply of forced air through conduit section 117 into the grounded ionizing zone 118, where contact with the high electric field surrounding the corona wire 122 imparts a positive charge to ionize the air. The ionized air molecules enter the mist of uncharged microparticles 125 in the microparticle charging zone 115 through conduit section 119. The alternating current field between the charging plates 120 and 121 spaced by about 1.5 mm and about 5 cm long, rapidly moves or vibrates the positive air ions into contact with the microparticles 125 to produce charged microparticles 126, which exit the nozzle 114 close to and in a direction normal to the surface area of the substrate. The nozzle opening has an exit gap of about 1/16^{th} inch and is about 5 cm wide, desirably larger than the substrate area of microlocations. A second stage fine filter, using direct current electrostatic precipitation plates, not shown, may be used in the area of the nozzle 114 to produce monodisperse microparticles. The nozzle may be designed in relation to the plate to be fixed in position or to move over the surface of the plate, directing the mist to different sets of microlocations. Thus, the nozzle may be elongated, rectangular, oval, etc.

The microlocations 129 in the substrate 130 are alternately negatively or positively charged to create an electric field for attracting the microparticles 125. Voltages can be +10V and −10V for microparticle attraction. Voltages up to about 50V may be employed, but will usually not be necessary.

Due to the dynamics of the impinging jet, the microparticles located near the centerline of the mist come very close to the substrate and are either strongly attracted or strongly repelled by the respective charged microlocations. Microparticles outside the centerline of the mist do not come sufficiently close to the substrate to experience any significant attraction or repulsion force and they are swept away by the air stream.

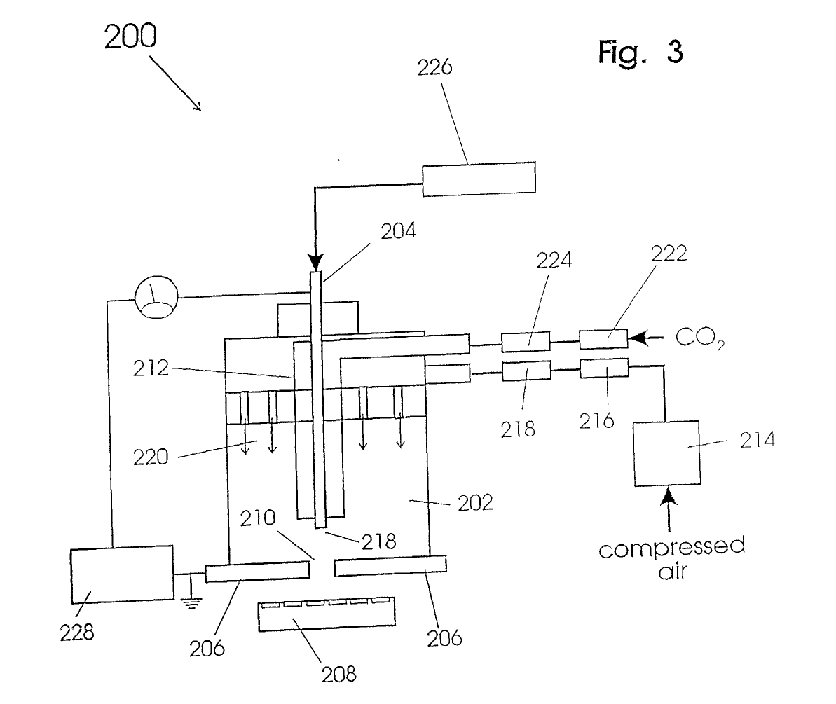

An alternative electrohydrodynamic device is depicted in FIG. 3. The device 200 has a spraying chamber 202 in the point-to-plate configuration with the capillary tube 204 facing the plate 206 and the microlocation substrate 208. An orifice 210 is located on the center of the plate 206 allowing the produced microparticles to impinge upon the substrate 208. The capillary tube is made of platinum with an ID of 81 μm and an OD of 224 μm. The distance from the tip 218 of the capillary tube 204 to the plate is about 5 mm. A coaxial tube 212 allows CO₂ to flow as a sheath surrounding the capillary tube for suppressing possible corona discharge. The compressed air is dried in dryer 214, measured with flowmeter 216 and then filtered with filter 218, before being introduced from above 220 in the spraying chamber 202. Similarly CO₂ is introduced into flow meter 222 and filtered by filter 224 before being introduced into coaxial tube 212. The compressed air serves to transport the particles through the orifice 210, The liquid is fed from a syringe pump 226. The flow rate is controlled by the syringe pump which is programmable.

A negative high voltage is applied to the plate 206 by voltage source 228. The capillary tube 204 is connected to an electrometer which is used to measure the spraying current. For monitoring the relationship between the measured current and the applied voltage, both signals are sent to an X-Y recorder. The size of the produced liquid microparticles is further reduced by the evaporation process. Microparticles of a size in the range of about 3 to 200 nm can be obtained. Flow rates are 2 lpm for the liquid and to 20 lpm for the sheath flow or 1.5 lpm and 15 lpm, respectively. Electrical conductivity is varied from about 15.6 to 8000 μΩ⁻¹ cm⁻¹. Feeding flow rates are from about 0.05 to 0.5 μl min⁻¹.

The arrays which are prepared, oligonucleotides and oligopeptides, may be used in a variety of ways for screening compounds. The oligonucleotide arrays, will have a plurality of the same oligonucleotide at each microlocation, the number being sufficient that in the presence of a specific binding compound, an homologous oligonucleotide or a compound binding to the oligonucleotide in the array, where the binding compound has a detectable label, e.g. a fluorophore or enzyme, the presence of the specific binding compound can be determined. Generally, there will be from about 10 to 10⁸ molecules present at a microlocation. The array is combined with a sample suspected of containing one or more specific binding compounds for at least some of the oligonucleotides present in the array, under conditions for binding of the sample compound(s) to the oligonucleotide. After washing away any non-specific binding compounds, the presence of a specific binding compound at a microlocation may be detected by means of the label. In some instances, one may use competitive labeled compounds, where the competitor competes with a compound in the sample for binding to the oligonucleotides in the array. The stronger the sample compound binds to the oligonucleotide, the less of the labeled competitor will be present. By using various optical devices one may read where the label is and determine what the oligonucleotide is by its location in the array.

Bulk solutions, which interact with all of the microlocations, may be removed in a variety of ways. For washes, the plate may be above a spray or other source of the wash solution, so that the wash solution will drain away from the microlocations. A wiper may be used to wipe away excess fluid from the plate. By inverting the plate, so that the microlocations are directed downwardly, liquid will drain away and a gas stream may be employed to remove the last vestiges of solvent.

Two methods of oligomeric sequence fabrication are discussed below using a memory chip to fabricate oligomeric arrays. In one method, a deprotection agent is supplied by the charged microparticle generator. In the other method, bases A, C, G, and T are supplied as nucleotide phosphoramidites by the charged microparticle generator.

In the first method, a RAM chip is provided that has a microwell formed in the passivation layers at each of the 500,000 cells of the RAM chip. The microwells have linking groups attached to the silica passivation layer, the metal electrode, the protective layer, or the electrically-conductive polymer layer within the microwells so that bases used to form oligomeric sequences may be attached to the microwells. Such linking groups are well-known and include those disclosed in U.S. Pat. No. 5,929,208. Each of the linking groups has the first nucleotide of the sequence to be formed attached to it. Each nucleotide is protected from further reaction by a protection group as discussed previously.

The reactant deposition system comprises a charged microparticle generator as illustrated in FIG. 1. The RAM chip is inserted into the reactant deposition system so that the nozzle 114 is positioned approximately 5-6 mm (¼ inch) below and normal to the surface of the RAM chip and facing the 500,000 microwells of the RAM chip. The RAM chip is electrically connected to the electronic system that controls the potential of the cells or microlocations by addressing the rows and columns of the cell array in the RAM chip, and the desired rows and columns are activated to supply the desired potential to the selected cells. The RAM chip is also heated to a temperature of approximately 104C.

Electrostatically charged droplets of deprotection agent (as discussed in U.S. Pat. No. 5,831,070 and 5,744,305, for example) having a diameter of about 1-5 micron and a mass of about 50 picogram each exit the nozzle 114 and are carried upwardly in a direction against the force of gravity by the gas stream. Cells having a lower potential than the potential of the electrostatically charged droplets attract the droplets of deprotection agent to those cells, while cells having a higher potential do not attract droplets and thus remain dry. Droplet deposition occurs over a sufficient period of time to partially fill the wells without overflowing them. Note that the liquid remains within the cells despite their inversion because of the surface, tension of the liquid within the cells, while the elevated substrate temperature helps to promote reaction as well as evolve some of the solvent from the deposited droplets. The RAM chip preferably has a layer whose thickness is selected to provide self-limiting deposition, as described previously.

Once a sufficient number of electrostatically-charged droplets of deprotection agent have been deposited in selected microwells, the RAM chip is removed from the charged microparticle generator. The deprotection reaction continues for a sufficient period of time to remove the protection agent from the protected bases within the microwells. The RAM chip is then rinsed and dried.

All microwells are then filled with a solution of a nucleoside phosphoramidite having the base that is to be added to the sequence. The unprotected bases that are attached to the microwells react with the nucleoside phosphoramidite and add the selected base to the sequence. The base as supplied in solution has a protection agent on it to prevent its further reaction with other bases in the solution within the microwell.

The RAM chip is then rinsed and dried and reinserted into the reactant deposition system. Cells are again selected by activating the desired rows and columns, and droplets of the deprotection agent are again attracted to selected microwells as described previously. Once the nucleotides are unprotected, the nucleotide containing the next base in the sequence is reacted with the growing oligonucleotide, and the process as described above is repeated until the desired sequences are formed at each of the cells of the array.

By activating selected rows and columns, individual cells are activated at any given time. Consequently, different oligonucleotide sequences can be grown at each of the cells if desired. A chip having 500,000 cells can therefore have up to 500,000 different oligonucleotide sequences on the chip.

The second method for growing desired oligonucleotide sequences at each cell of the RAM chip involves selective deposition of electrostatically charged droplets of nucleoside phosphoramidite.

Again, a RAM chip is provided that has a microwell formed in the passivation layers at each of the 500,000 cells of the RAM chip. The microwells have linking groups attached to the silica passivation layer, the metal electrode, the protective layer, or the electrically-conductive polymer layer within the microwells so that nucleotides used to form oligomeric sequences may be added to the growing oligomeric chains. Each of the linking groups has the first nucleotide of the sequence to be formed attached to it, but each nucleotide is unprotected, i.e. the nucleotide does not have a protection group as discussed previously.

The RAM chip is inserted into the reactant deposition system so that the nozzle 114 is positioned approximately 5-6 mm (¼ inch) below and normal to the surface of the RAM chip and facing the 500,000 microwells of the RAM chip. The RAM chip is electrically connected to the electronic system that controls the potential of the cells or microlocations by addressing the rows and columns of the cell array in the RAM chip, and the desired rows and columns are activated to supply the desired potential to the selected cells. The RAM chip is also heated to a temperature of approximately 104C.

Electrostatically charged droplets of the first nucleoside-phosphoramidite having a diameter of about 1-5 micron and a mass of about 50 picogram each exit the nozzle 114 and are carried upwardly in a direction against the force of gravity by the gas stream. Cells having a lower potential than the potential of the electrostatically charged droplets attract the nucleoside-phosphoramidite droplets to those cells, while cells having a higher potential do not attract droplets and thus remain dry. Droplet deposition occurs over a sufficient period of time to partially fill the wells without overflowing them. The added nucleotide has protection groups on it to prevent further reactions from occurring.

Once a sufficient number of electrostatically-charged droplets of the first nucleoside-phosphoramidite have been deposited in selected microwells, the electronic system selects a different set of microwells and supplies a potential to selected cells. Electrostatically-charged droplets of a second nucleoside-phosphoramidite are formed by the microparticle generator, and these droplets are carried by the gas stream to the microwells of the RAM chip. The selected cells attract droplets of the second nucleoside-phosphoramidite, while the cells not selected do not attract the droplets from the aerosol. This added nucleotide has protection groups on it to prevent further reactions from occurring. Once a sufficient period of time has passed, deposition of the second nucleoside-phosphoramidite is halted.

Again, the electronic system selects a different set of microwells and supplies a potential to selected cells. Electrostatically-charged droplets of a third nucleoside-phosphoramidite are formed by the microparticle generator, and these droplets are carried by the gas stream to the microwells of the RAM chip. The selected cells attract droplets of the third nucleoside-phosphoramidite, while the cells not selected do not attract the droplets from the aerosol. The added nucleotide also has protection groups on it to prevent further reactions from occurring. Deposition stops once a sufficient amount of the third nucleoside-phosphoramidite is deposited in the selected microwells.

The electronic system again selects a different set of microwells and supplies a potential to selected cells. Electrostatically-charged droplets of the fourth nucleoside-phosphoramidite are formed by the microparticle generator, and these droplets are carried by the gas stream to the microwells of the RAM chip. The selected cells attract droplets of the fourth nucleoside-phosphoramidite, while the cells not selected do not attract the droplets from the aerosol. The added nucleoside-phosphoramidite has protection groups on it to prevent further reactions from occurring. Deposition stops once a sufficient amount of the fourth nucleoside-phosphoramidite is deposited in the selected microwells.

The RAM chip is then removed from the charged microparticle generator. Once the nucleoside phosphoramidite solutions within the microwells react with the oligomeric sequences attached to the chip, the RAM chip is rinsed and dried.

Each of the microwells is then filled with the deprotection agent. The deprotection reaction continues for a sufficient period of time to remove the protection agent from all of the protected bases within the microwells. The RAM chip is then rinsed and dried.

The process is then repeated. Each of the four protected nucleoside-phosphoramidites is individually deposited by selecting the desired rows and columns and applying a potential to selected cells. Once the shortest oligomeric sequence desired is completely formed, those cells containing these oligomeric sequences will not be selected again for further deposition. Consequently, only those cells in which further deposition is to occur will be selected during the deposition sequence, and cells on the chip can have sequences that vary in length as well as in base sequence from one another.

By activating selected rows and columns, individual cells are activated at any given time. Consequently, different oligonucleotide sequences can be grown at each of the cells if desired. A chip having 500,000 cells can therefore have up to 500,000 different oligonucleotide sequences of any desired length on the chip.

The system of this invention differs markedly from the deposition system described in U.S. Pat. No. 5,965,452 and 5,929,208, for example. In the '452 and '208 patents, the system utilizes an electrophoretic electrode to attract ionically charged species that are present within a solution. Electrophoretic transport generally results from applying a voltage which is sufficient to permit electrolysis and ion transport within a solution in the system. The '208 patent explains that a complete sequence of interest is transported to an electrode by electrophoretic transport, where the complete sequence reacts with the functionalized surface to provide the sequence of interest attached at that location. The microwells are therefore filled by e.g. flooding the surface of the substrate with the desired ionic sequence and attracting the ionic sequence using electrophoretic transport (see the '208 patent, col. 15 lines 24-63). The system of the '452 and '208 patents cannot, therefore, attract species within a solution that are not ionic. Further, the system of the '452 and '208 patents utilizes a different mechanism, electrophoretic transport in a solution and not electrostatic attraction of droplets, to attach oligomeric sequences to a substrate.

The genetic array on a memory chip of this invention also differs from the array of the '452 patent. The substrate of the '452 patent requires a separate line attached to its electrophoretic electrode. The electrical circuit through the line and to the electrophoretic electrode is controlled by two transistors, and an analog signal is supplied to the electrophoretic electrode. The chip of this invention does not require a separate line with an analog signal to an electrode. The electrode on the chip of this invention is attached to or is part of the circuitry that controls a memory cell, and therefore the electrode is or is in electrical connection with a source, drain, gate, cathode, anode, or floating gate on the substrate.

The reactants for use in this invention have been described above as a protected nucleoside-phosphoramidite. However, the reactants could be e.g. a desired sequence of nucleotides in a phosphoramidite form, or the reactants may be a solid particulate in suspension or even may be dry solid particulate that is electrostatically charged and carried in a gaseous stream to deposit at selected cells of an array such as a RAM chip by electrostatic attraction as described previously.

An additional charged microparticle generator and method of reactant deposition that is useful in the practice of this invention is disclosed in PCT publication WO 98/58745, the contents of which are incorporated by reference in their entirety as if fully put forth herein. This reactor can utilize an x-y positioning stage to move the substrate beneath multiple deposition nozzles, for instance.

Various detection systems may be used, such as CCDs, fluorimeters, spectrophotometers, gas chromatography, mass spectrometry, and the like. With mass spectrometry one may be able to avoid having a labeled compound present.

A particularly useful substrate of this invention has a light detector formed beneath each cell of the cell array. A photodiode or charge-coupled device as known in the art can be formed beneath each cell as the cell array is formed on the substrate. Greater sensitivity to fluorescence is obtained by placing a light detector beneath each cell, with each detector detecting approximately 50% of the available light rather than the 1-2% as is commonly obtained when the light detector is positioned above the substrate during use. Consequently, a RAM or ROM memory chip that has been custom-fabricated to also have a light detector beneath each cell in its array is a particularly preferred substrate for forming an array of oligomers. Further details of a light detector suitable for this application are disclosed in U.S. Pat. No. 5,965,452.

Alternatively, a light source such as a light emitting diode may be positioned beneath each cell on the substrate to illuminate the contents of each microwell. A light detector may be positioned above the substrate to detect fluorescence. Or, a light detector may be positioned beneath each cell and separated from the detector by an opaque wall of material formed by, e.g., implanting a material that changes the refractive index between the detector and LED or by etching a well and depositing a material having a suitable refractive index to prevent light from the LED from shining directly onto the detector.

The oligopeptides may be used for assays in an analogous manner, except that the oligopeptides will act as epitopic sites. One can then screen candidate compounds for their binding affinity to the different oligopeptides present in the array to determine which compounds have an affinity for a particular oligopeptide. By having known labeled binding compounds, one can provide a competition between the known labeled compounds and the candidate compounds under binding conditions. The absence of the labeled compound at a microlocation would be indicative of the binding of the candidate compound.

Assays may be carried out where the oligomers may be tested for their enyzme activation or inhibition. One could use a bulk solution of enzyme and substrate at the different microlocations. Where the product of the enzymatic reaction provides a detectable signal, the activation or inhibition of the oligomer at the microlocation could be determined.

Other assays can be performed, where one or more reagents are directed to a microlocation by mist transfer. One could direct different test compounds to different microlocations and then direct the appropriate agents for the assay to the individual microlocations. For example, if one were interested in a number of compounds for binding affinity to a variety of proteins, one could direct different proteins to different microlocations. The surface of the microlocation would be such that the protein would non-covalently bind or one could functionalize the microlocation surface, so as to form covalent bonds with the protein. After the protein was present, one would then direct different compounds with labeled competitors to different microlocations by mist transfer. After incubating, one would wash away non-specifically bound compound and determine the amount of labeled competitor present. The less of the label present, the stronger the binding affinity.

By employing the use of charged mist microparticles with microlocations of a substrate, one can perform a number of different operations, rapidly, efficiently and using very small amounts of chemical moieties, which may be only available with difficulty and expensive. By having self-addressable microlocations, one does not need to code for chemical events, but may keep a computer log, which not only controls the different chemical moieties and the microlocations to which they are directed, but also records the events. Therefore, upon completion of the operation, one has a computer record of what has occurred at each location. One can direct specific compounds of interest to a particular location or prepare oligomers at specific locations and then perform operations on these entities at the microlocations. In this way, one can direct particular chemical moieties to predetermined microlocations and then determine the effect of the combining of the chemical moieties at the specified microlocation. By using mists, one may have redundancy, if one wishes, so that the values from different microlocations which have undergone the same operation may be compared. Since the operation is computer controlled, all of the events, are accurately recorded without manual intervention.

All publications and patent applications mentioned in this specification are indicative level of skill of those skilled in the art to which this invention pertains. All publications and patent applications are herein incorporated by reference to the same extent as if each individual publication or patent application was specifically and individually indicated to be incorporate by reference. This application also incorporates by reference in its entirety herein the application entitled “Multiplexed Generation of Chemical or Physical Events,” Inventor: Steve Herrick, filed Aug. 7, 1999.

The invention now having been fully described, it will be apparent to one of ordinary skill in the art that many changes and modifications can be made thereto without departing from the spirit or scope of the appended claims.

What is claimed is:

1. A method of performing at least one operation at a plurality of microlocations having volume capacities of less than about 5 ml, employing a charged microparticle mist and a substrate having controlled microelectrodes at selected voltages, such that the potential gradient between said microelectrodes and charged microparticles in said mist causes said microparticles to be attracted to a selected first group of microlocations and repelled by a selected second group of microlocations, said method comprising:
(1) forming a mist of unipolar microparticles comprising at least one chemical moiety for performing said operation;
(2) directing said mist into proximity to said microlocations, wherein microparticles of lower potential to said microlocations are captured by said microlocation;
repeating steps (1) and (2) as required for the same or different microlocations;
whereby said operations are performed at at least a portion of said plurality of microlocations.

2. A method according to claim 1, wherein said operation is the preparation of an oligonucleotide, wherein in step (1) said chemical moiety is a nucleotide derivative for addition to a prior bound nucleotide for preparation of said oligonucleotide, said nucleotide derivative comprising a blocking group, and after completing steps (1) and (2), a chemical moiety is added for deblocking or said blocking group is removed photolytically, thermally or electrolytically.

3. A method according to claim 1, wherein said operation is the preparation of an oligonucleotide, wherein in step (1) said chemical moiety is a nucleotide derivative for addition to a prior bound nucleotide for preparation of said oligonucleotide, said nucleotide derivative comprising a blocking group, each of said microlocations has a terminal deoxytransferase for adding said nucleotide derivative, and after completing steps (1) and (2), a chemical moiety is added for deblocking or said blocking group is removed photolytically, thermally or electrolytically.

4. A method according to claim 1, wherein said operation is the preparation of an oligopeptide, and wherein in step (1) said chemical moiety is an amino acid derivative for addition to a prior amino acid for preparation of said oligopeptide comprising a blocking group, and after completing steps (1) and (2), a chemical moiety is added for deblocking or said blocking group is removed photolytically, thermally or electrolytically.

5. A method according to claim 1, including the additional step of washing said microlocations before repeating steps (1) and (2).

6. A method according to claim 1, wherein in step (1) said chemical moiety is a synthon in a preparative synthesis.

7. A method according to claim 1, wherein said mist is applied perpendicularly to said substrate.

8. A method according to claim 1, wherein said mist is applied parallel to said substrate.

9. A method according to claim 1, wherein said mist is applied at other than parallel or normal to said substrate.

10. A method of performing at least one operation at a plurality of microlocations having volume capacities of less than about 500μ, employing a charged microparticle mist and a substrate having controlled microelectrodes at selected voltages, such that the potential gradient between said microelectrodes and charged microparticles in said mist causes said microparticles to be attracted to a selected first group of microlocations:
(1) forming a mist of unipolar charged microparticles comprising at least one chemical moiety for performing said operation;
(2) directing said mist into proximity to said microlocations, wherein microparticles of lower potential to said microlocations are captured by said microlocation;
repeating steps (1) and (2) as required for the same or different microlocations;
recording said microlocation and said at least one chemical moiety at each stage for a history of the operation at each microlocation;
whereby said operations are performed at at least a portion of said plurality of microlocations.

11. A method according to claim 10, wherein said operation is the preparation of an oligomer selected from the group consisting of oligonucleotides and oligopeptides, wherein in step (1) said chemical moiety comprises a blocking group and is a monomer selected from the group consisting of a nucleotide derivative or an amino acid derivative for addition to a prior monomer for preparation of said oligomer and after completing steps (1) and (2), a chemical moiety is added for deblocking or said blocking group is removed photolytically, thermally or electrolytically, whereby an array of oligomers is produced.

12. A method according to claim 11, wherein said array is contacted with at least one compound to determine binding of said compound to an oligomer in said array.

13. A method according to claim 11, wherein said array is contacted with at least one compound to determine binding of said compound to an oligomer and a labeled competitive compound.

14. A method according to claim 11, wherein said microlocations contain beads on which said at least one chemical moiety becomes bonded.

15. A method of performing at least one operation at a plurality of micro locations having volume capacities of less than about 5 ml, employing a charged microparticle mist and a substrate having controlled microelectrodes at selected voltages, such that the potential gradient between said microelectrodes and charged microparticles in said mist causes said microparticles to be attracted to a selected first group of microlocations
(1) forming a mist of aqueous unipolar charged microparticles comprising at least one chemical moiety for performing said operation;
(2) directing said mist into proximity to said microlocations, wherein microparticles of lower potential to said microlocations are captured by said microlocation;
repeating steps (1) and (2) as required for the same or different microlocations;
recording said microlocation and said at least one chemical moiety at each stage for a history of the operation at each microlocation;
whereby said operations are performed at at least a portion of said plurality of microlocations.

16. A device capable of producing a plurality of compounds in close proximity at microlocations on a substrate, said device comprising:
an insulating substrate;
a plurality of microlocations, each microlocation comprising a microelectrode connected to a wire network for connection to a computer, whereby said computer controls the voltage and potential at each microelectrode to cause nearby micro electrodes to have a different potential;
insulation between each of said microlocations;
means for producing unipolar charged microparticles comprising at least one chemical moiety; and
means for directing said microparticles from said microparticle producing means to said microlocations in liquid form and delivering said liquid microparticles to said microlocations.

17. A device according to claim 16, further comprising a chemically reactive moiety in each microlocation for reacting with said chemical moiety.

18. A device according to claim 16, wherein said insulating substrate and insulation is silicon dioxide or silicon nitride.

19. A device according to claim 16, wherein said microlocations have volumes of less than about 500 μl.

20. A device according to claim 16, wherein the density of microlocations is in the range of about 100 to 106.

21. A device according to claim 16, comprising means for producing monodispersed microparticles.

22. A device according to claim 16, wherein said means for producing said charged microparticles is selected from the group consisting of an aerosolizer in combination with corona discharge, an aerosolizer in combination with ionizing radiation, or electrohydrodynamic generation.

23. A device capable of producing a plurality of compounds in close proximity at microlocations on a substrate, said device comprising:
an insulating substrate;
a plurality of microlocations, each microlocation comprising a microelectrode connected to a wire network for connection to a computer, whereby said computer controls the voltage and potential at each microelectrode to cause nearby microelectrodes to have different potential;
insulation between each of said micro locations;
different chemical moieties or beads in different microlocations;
means for producing charged microparticles comprising at least one chemical moiety; and
means for directing said microparticles from said microparticle producing means to said microlocations in liquid form and delivering said liquid microparticles to said microlocations.

24. A device according to claim 23, wherein in said different microlocations are different chemical moieties and said different chemical moieties are different oligomers.

25. A device according to claim 23, wherein in said different microlocations are beads.

26. A device according to claim 23, wherein said microlocations have a volume of less than about 500 μl.

27. A substrate for forming an array of polymeric sequences, said substrate comprising a plurality of cells arranged in an addressable array and having row address lines and column address lines that are configured to address each cell of the addressable array, each of said cells having an electrode that is part of or is electrically attached to the circuitry that controls the addressing of the addressable array of cells, and each of said cells being individually positioned at a microlocation on said substrate and having a microwell at said microlocation having a sufficient depth that an electric field can be established by said electrode at said microlocation, said electric field having a sufficient strength to attract an electrostatically-charged microparticle from a gaseous carrier into said microwell.

28. A substrate in accordance with claim 27 and further comprising a plurality of oligomeric sequences attached to the substrate at said microlocation and within said microwell.

29. A substrate in accordance with claim 28 wherein a first cell of said plurality of cells contains a first oligomeric sequence within a first microwell and a second cell of said plurality of cells contains a second oligomeric sequence within a second microwell, said second oligomeric sequence being different from said first oligomeric sequence.

30. A substrate in accordance with claim 29 wherein said first oligomeric sequence comprises a first oligonucleotide sequence, and wherein said second oligomeric sequence comprises a second oligonucleotide sequence.

31. A substrate in accordance with claim 30 wherein said plurality of cells comprises at least about 64,000 cells.

32. A substrate in accordance with claim 27 wherein said plurality of cells comprises at least about 64,000 cells.

33. A substrate in accordance with claim 31 wherein said plurality of cells comprises at least about 256,000 cells.

34. A substrate in accordance with claim 27 wherein said electrode has a layer that forms part of the microwell, the thickness of said layer being sufficient to regulate the number of electrostatically charged microparticles that deposit in said microwell.

35. A reactant deposition system for making a substrate containing oligomeric sequences at microlocations on said substrate, said reactant deposition system comprising a charged microparticle generator that generates electrostatically charged droplets having a first potential; a memory chip having a plurality of cells and a plurality of microwells that are individually positioned above at least some of said plurality of cells; and an electronic system that places a second potential on selected cells and a third potential on unselected cells of said plurality by activating rows and columns in the memory chip and placing said second potential at said selected cells and said third potential at said unselected cells, said second potential being sufficient to attract said electrostatically charged droplets to microwells above said selected cells and said third potential being sufficient to prevent said electrostatically charged droplets from depositing in microwells above said unselected cells; and wherein the microparticle generator is configured to produce a moving aerosol of said electrostatically charged droplets that is directed toward said substrate, which droplets deposit within microwells of said selected cells and which droplets do not deposit within microwells of said unselected cells.

36. The reactant deposition system of claim 35 wherein said plurality of cells of said memory chip comprises at least about 64,000 cells.

37. The reactant deposition system of claim 35 wherein said electrostatically charged droplets comprise a deprotection reagent.

38. The reactant deposition system of claim 35 wherein said electrostatically charged droplets comprise a base-phosphoramidite useful in forming oligonucleotide sequences.

39. The reactant deposition system of claim 35 further comprising four liquid storage vessels configured to contain respectively an adenosine-containing nucleotide phosphoramidite solution useful in forming oligonucleotide sequences; a thymine-containing nucleotide phosphoramidite solution useful in forming oligonucleotide sequences; a guanine-containing nucleotide phosphoramidite solution useful in forming oligonucleotide sequences; and a cytosine-containing nucleotide phosphoramidite solution useful in forming oligonucleotide sequences.

40. A method of making an oligomeric array comprising
a) forming an aerosol of electrostatically-charged droplets;
b) directing said aerosol at a surface of a memory chip containing a plurality of cells, each of said cells being addressed individually by electrical signals applied to a row line and a column line and each of said cells individually having a microwell positioned above said cells in said surface of the memory chip;
c) addressing a first cell of said plurality of cells, thereby applying a potential to said first cell that is sufficient to attract said electrostatically-charged droplets to its corresponding microwell; and
d) depositing a desired number of said electrostatically-charged droplets in said corresponding microwell.

41. The method of claim 40 wherein the act of depositing the desired number of said electrostatically-charged droplets in said corresponding microwell is controlled by selecting the thickness of the floor of said microwell to provide a desired field strength within said microwell.

42. The method of claim 40 wherein said electrostatically-charged droplets comprise a deprotecting agent.

43. The method of claim 40 wherein said electrostatically-charged droplets comprise a base nucleotide phosphoramidite useful in forming an oligonucleotide sequence.

44. The method of claim 40 wherein a number of said plurality of cells is addressed simultaneously, and said number is greater than one and less than the total number of cells in said plurality of cells.

45. The method of claim 44 wherein a first cell of said number of cells addressed simultaneously contains a first oligomeric sequence, and a second cell of said number of cells addressed simultaneously contains a second oligomeric sequence different from said first oligomeric sequence.

46. The method of claim 40 further comprising addressing a second cell of said plurality of cells subsequent to selecting said first cell and further depositing a desired number of said electrostatically-charged droplets in its corresponding microwell.

47. The method of claim 46 wherein the electrostatically-charged droplets deposited at said first cell comprise a first reactant, the electrostatically-charged droplets deposited at said second cell comprise a second reactant, and the first reactant differs from the second reactant.

48. A microarray of oligonucleotides made by the method of any of claims 1.

49. A microarray of oligonucleotides made by the method of claim 10.

50. A microarray of oligonucleotides made by the method of claim 15.

51. A microarray of oligonucleotides made by the method of claim 40.
